(19) United States
(12) Patent Application Publication
(10) Pub. No.: US 2026/0258435 A1
(43) Pub. Date: Sep. 3, 2026

(54) USE OF SOYBEAN C2H2 TYPE ZINC FINGER PROTEIN TRANSCRIPTION FACTOR GMZFP7 AND/OR GENE THEREOF IN REGULATING ISOFLAVONES

(71) Applicant: INSTITUTE OF CROP SCIENCES, CHINESE ACADEMY OF AGRICULTURAL SCIENCES, Beijing (CN)

(72) Inventors: Junming SUN, Beijing (CN); Bin LI, Beijing (CN); Shengrui ZHANG, Beijing (CN); Yue FENG, Beijing (CN); Jing LI, Beijing (CN); Ruili PEI, Beijing (CN); Ling TIAN, Beijing (CN)

(73) Assignee: INSTITUTE OF CROP SCIENCES, CHINESE ACADEMY OF AGRICULTURAL SCIENCES, Beijing (CN)

(21) Appl. No.: 18/867,015

(22) PCT Filed: May 16, 2023
(86) PCT No.: PCT/CN2023/094563

§ 371 Date: Nov. 19, 2024

(30) Foreign Application Priority Data
Aug. 19, 2022 (CN) 202211003501.6

(51) Int. Cl. C12N 15/82

(52) U.S. Cl. CPC C12N 15/8243

(57) ABSTRACT

A use of a soybean C2H2 type zinc finger protein transcription factor GmZFP7 with the amino acid sequence as shown in SEQ ID NO: 2, or a gene GmZFP7 with a gene accession number of Gene ID: 100792169 in regulating isoflavones is provided. Also provided is a method for regulating isoflavones, which increases or reduces a total isoflavone content in a plant by way of overexpression, silencing, knockdown or knockout of the transcription factor gene GmZFP7.

### TECHNICAL FIELD

The present disclosure belongs to the field of plant genetic engineering, and in particular to a use of soybean C2H2 type zinc finger protein transcription factor GmZFP7 and/or a gene thereof in regulating isoflavones.

### BACKGROUND TECHNOLOGY

Isoflavones are important secondary metabolites synthesized in plants, especially in legumes. Isoflavones play an important role in animals and plants. In animals, especially humans, their chemical structures are similar to that of estrogen, and thus they have estrogen-like activity. Isoflavones play important roles in fighting cancers, relieving osteoporosis, reducing cardiovascular and cerebrovascular diseases, preventing and curing menopausal syndrome in women, and the like. In plants, isoflavones play important roles in resisting pathogenic bacteria infection, acting as signal molecules to induce soybean nodulation and ensuring normal growth and development of plants. Due to an enormous application value of isoflavones in foods and health products, they have received widespread attention. Soybean is a main natural source of isoflavones, which makes a cultivation of special soybean varieties with high isoflavone content become one of the main objectives of soybean nutritional quality breeding.

Genetic engineering is an effective means to improve crop traits. At present, a use of genetic engineering to increase the content of soybean isoflavones is mainly achieved by modifying structural genes of isoflavone synthases and key enzyme genes in an isoflavone competition pathway. However, metabolism and synthesis pathways of important secondary metabolites as flavonoids and anthocyanins in transgenic plants are blocked, resulting in problems such as difficulty in survival of the transgenic plants. In addition, some MYB transcription factors have been identified to regulate expression levels of key enzyme genes in the isoflavone synthesis pathway, thereby affecting an accumulation level of soybean isoflavones. However, the transcription factor genes identified so far that regulate isoflavone content are still very few, and there is a lack of in-depth functional mechanism research.

GmZFP7 (gene locus number: Glyma.20G012700, gene accession number Gene ID: 100792169) is a gene encoding soybean zinc finger protein 7, and the protein encoded thereby is a soybean C2H2 type zinc finger protein transcription factor GmZFP7. A functional identification of this gene and protein has not been reported in the art, and a relationship between this gene, protein and isoflavone content has never been reported in the art.

### CONTENT OF THE INVENTION

Based on the above-mentioned blanks of the prior art in the art, the present disclosure provides a use of a soybean C2H2 type zinc finger protein transcription factor GmZFP7 with an amino acid sequence as shown in SEQ ID NO.2, or an encoding gene thereof with a gene accession number of Gene ID: 100792169 in regulating isoflavones.

The technical solution of the present disclosure is as follows:

The use of a soybean C2H2 type zinc finger protein transcription factor GmZFP7 with the amino acid sequence as shown in SEQ ID NO.2 in regulating isoflavones is provided.

A gene accession number of an encoding gene GmZFP7 of the soybean C2H2 type zinc finger protein transcription factor GmZFP7 is Gene ID: 100792169.

A nucleotide sequence of the gene GmZFP7 of the soybean C2H2 type zinc finger protein transcription factor GmZFP7 is as shown in SEQ ID NO.1.

The regulating isoflavones refers to increasing or reducing an isoflavone content in a plant by increasing or decreasing a level of the soybean C2H2 type zinc finger protein transcription factor GmZFP7 in a plant body, or by way of overexpression, silencing, knockdown or knockout of the gene GmZFP7 of the soybean C2H2 type zinc finger protein transcription factor GmZFP7 in the plant body; and
the plant is selected from the group consisting of soybeans and tobaccos.

A use of a gene GmZFP7 with a gene accession number of Gene ID: 100792169 in regulating isoflavones is provided.

A gene locus number of the gene GmZFP7 is Glyma.20G012700; and a nucleotide sequence of the gene GmZFP7 is as shown in SEQ ID NO.1.

The gene GmZFP7 regulates isoflavones by activating an expression of a gene GmIFS2 encoding isoflavone synthase 2, and/or inhibiting an expression of a gene GmF3H1 encoding flavanone-3-hydroxylase 1.

The regulating isoflavones refers to increasing or reducing an isoflavone content in a plant; and preferably, the plant is selected from the group consisting of soybeans and tobaccos.

Also provided is a method for regulating isoflavones, which method regulates the isoflavones by regulating a level of a soybean C2H2 type zinc finger protein transcription factor GmZFP7 with an amino acid sequence as shown in SEQ ID NO.2, and/or by regulating an expression of a gene GmZFP7 with a gene accession number of Gene ID: 100792169.

The regulation of the level of the soybean C2H2 type zinc finger protein transcription factor GmZFP7 with the amino acid sequence as shown in SEQ ID NO.2, and/or the regulation of the expression of the gene GmZFP7 with the gene accession number of Gene ID: 100792169 are/is achieved by way of overexpression, silencing, knockdown or knockout of the gene GmZFP7 encoding the soybean C2H2 type zinc finger protein transcription factor GmZFP7.

The overexpression refers to introducing a recombinant overexpression vector PTF101-GmZFP7-GFP or pGGP-GmZFP7 into a plant, wherein the recombinant overexpression vector is obtained by cloning the gene GmZFP7 to an expression vector PTF101-GFP or pGGP; preferably, nucleotide sequences of primers used for cloning the gene GmZFP7 to the expression vectors are as shown in SEQ ID NO.5-6;
the silencing refers to introducing a recombinant silencing expression vector pGGP-GmZFP7-RNAi into a plant, wherein the recombinant silencing expression vector is obtained by cloning an RNAi sequence targeting the gene GmZFP7 into the vector pGGP;
preferably, nucleotide sequences of primers used for cloning the RNAi sequence targeting the gene GmZFP7 into the vector pGGP are as shown in SEQ ID NO.7-10;
the knockout refers to introducing a JRH0645-GmZFP7 gene editing vector into a plant;
preferably, the JRH0645-GmZFP7 gene editing vector is constructed by using PCR to implement a series connection of a U6 promoter, gRNA targeting GmZFP7 and gRNA scaffold and then inserting same at an XbaI cleavage site in an original vector JRH0645 (CaMV35s: Cas9); and preferably, a target sequence of the gRNA is as shown in SEQ ID NO.31.

The present disclosure discloses the use of the soybean C2H2 type zinc finger protein transcription factor GmZFP7 and its encoding gene GmZFP7 (gene locus number: Glyma.20G012700) in regulating soybean isoflavone content. The transcription factor GmZFP7 has an amino acid sequence as shown in SEQ ID No.2. The open reading frame of the gene has a DNA sequence as shown in SEQ ID No.1. The transcription factor GmZFP7 provided by the present disclosure has bifunctional transcription factor activity, which can activate the expression of the key enzyme isoflavone synthase 2 (IFS2) in a soybean isoflavone synthesis pathway, while inhibiting the expression of the key enzyme flavanone 3-hydroxylase 1 (F3H1) in a flavonol synthesis pathway.

Overexpression of the open reading frame of the gene encoding the transcription factor in soybean hairy roots can significantly increase a total isoflavone content in the soybean hairy roots; and inhibition of the expression of the gene encoding the transcription factor can significantly reduce the total isoflavone content in the soybean hairy roots. In stable transgenic soybean plants, the total isoflavone contents in leaves and seeds of transgenic plants overexpressing the gene of the transcription factor are all significantly increased, and the total isoflavone contents in leaves and seeds of mutant plants with the gene knocked out by gene editing are significantly reduced. The present disclosure has important application value for cultivating new soybean varieties with different levels of isoflavone content.

### DESCRIPTION OF THE DRAWINGS

FIG. 1 is a schematic structural diagram of a gene overexpression vector pGGP-GmZFP7 based on GFP detection in Section 3 Experimental Methods of Part One Materials and Methods in an experimental example according to the present disclosure.

FIG. 2 is a schematic structural diagram of a gene silencing vector pGGP-GmZFP7-RNAi based on GFP detection in Section 3 Experimental Methods of Part One Materials and Methods in an experimental example according to the present disclosure.

FIG. 3 is a schematic structural diagram of a plant expression vector PTF101-GmZFP7-GFP in Section 3 Experimental Methods of Part One Materials and Methods in an experimental example according to the present disclosure.

FIG. 4 is a schematic structural diagram of a plant expression vector pGreen-promoter-LUC in Section 3 Experimental Method of Part One Materials and Methods in an experimental example according to the present disclosure.

FIG. 5 is a diagram of a gene editing vector JRH0645-GmZFP7 in Section 3 Experimental Methods of Part One Materials and Methods in an experimental example according to the present disclosure.

FIG. 6 is column diagrams of isoflavone contents and relative expression levels of GmZFP7 in LHD hairy roots in Section 1 of Part Two Experimental Results in an experimental example according to the present disclosure, where (A) represents the relative expression levels of GmZFP7 in the hairy roots, and (B) represents the relative contents of total isoflavones in GmZFP7 overexpressed hairy roots and GmZFP7 silenced hairy roots.

FIG. 7 is a vector schematic diagram and induction level column diagrams of GmZFP7 inducing and regulating GmIFS2 and GmF3H1 promoter activities in Section 2 of Part Two Experimental Results in an experimental example according to the present disclosure, where (A) represents a schematic diagram of a tobacco transient expression vector, and (B) represents column diagrams of the induction levels of GmZFP7 on GmIFS2 and GmF3H1 promoter activities.

FIG. 8 is the acquisition and detection results of GmZFP7 overexpression transgenic lines in Section 3.1 of Part Two Experimental Results in an experimental example according to the present disclosure, where (A) represents lines with GmZFP7 overexpressed and a control line; (B) represents the BAR test strip detection results of plants with GmZFP7 overexpressed, which shows that the three lines with GmZFP7 overexpressed, i.e., GmZFP7-OE1, GmZFP7-OE2 and GmZFP7-OE3, all have positive bands for herbicide resistance genes; and (C) is a diagram showing the effect of the resistance of transgenic leaves to glufosinate, in which the leave with positive result is from the line GmZFP7-OE2 with GmZFP7 overexpressed. The leaves of GmZFP7-OE1 and GmZFP7-OE3 have a phenotype similar to that of GmZFP7-OE2.

FIG. 9 is the results of the phenotypic identification of transgenic lines with GmZFP7 overexpressed in Section 3.2 of Part Two Experimental Results in an experimental example according to the present disclosure, where (A) represents a column diagram showing the gene expression level of GmZFP7 in leaves of the overexpressed lines; (B) represents a column diagram showing the changes in isoflavone contents in the leaves of the lines with GmZFP7 overexpressed; (C) represents a column diagram showing the changes in isoflavone content in seeds of the lines with GmZFP7 overexpressed; and (D) represents column diagrams showing the changes in the expression levels of GmIFS2 and GmF3H1 in the leaves of the overexpressed lines.

FIG. 10 is the screening process and results of CRISPR/Cas9-mediated plants with GmZFP7 knocked out in Section 3.3 of Part Two Experimental Results in an experimental example according to the present disclosure, where (A) represents the position and sequence of gRNA in GmZFP7; (B) shows a PCR detection electrophoresis map for the CRISPR/Cas9 sequence; (C) shows Williams82 control and mutant seedlings with GmZFP7 knocked out; and (D) shows the screening for a mutant with GmZFP7 knocked out.

FIG. 11 is the results of the phenotypic identification of a GmZFP7 mutant in Section 3.4 of Part Two Experimental Results in an experimental example according to the present disclosure, where (A) represents a column diagram showing the total isoflavone content in the leaves of the Gmzfp7 mutant; (B) represents a column diagram showing the total isoflavone content in the seeds of the Gmzfp7 mutant; (C) represents a column diagram showing the changes in the expression levels of GmZFP7 in the leaves of the Gmzfp7 mutant; and (D) represents column diagrams showing the changes in the expression levels of GmIFS2 and GmF3H1 in the leaves of the Gmzfp7 mutant.

### SPECIFIC IMPLEMENTATIONS

The details of the present disclosure will be further specifically described below in conjunction with specific examples and experimental examples, but the protection scope of the present disclosure is not limited thereto.

First Group of Examples: New application of transcription factor GmZFP7 provided by the present disclosure The examples in this group provide a use of a soybean C2H2 type zinc finger protein transcription factor GmZFP7 with an amino acid sequence as shown in SEQ ID NO.2 in regulating isoflavones.

In a specific example, a gene accession number of an encoding gene GmZFP7 of the soybean C2H2 type zinc finger protein transcription factor GmZFP7 is Gene ID: 100792169.

In a more specific example, a nucleotide sequence of the gene GmZFP7 encoding the soybean C2H2 type zinc finger protein transcription factor GmZFP7 is as shown in SEQ ID NO.1.

Preferably, the regulating isoflavones refers to increasing or reducing an isoflavone content in a plant by increasing or decreasing a level of the soybean C2H2 type zinc finger protein transcription factor GmZFP7 in a plant body, or by way of overexpression, silencing, knockdown or knockout of the gene GmZFP7 encoding the soybean C2H2 type zinc finger protein transcription factor GmZFP7 in the plant body.

Preferably, the plant is selected from the group consisting of soybeans and tobaccos.

In some specific examples, regulating isoflavones refers to increasing or decreasing the content of isoflavones in roots, leaves and seeds of a plant.

Second Group of Examples: New application of gene GmZFP7 provided by the present disclosure

The examples in this group provide a use of a gene GmZFP7 with a gene accession number of Gene ID: 100792169 in regulating isoflavones.

In some examples, a gene locus number of the gene GmZFP7 is Glyma.20G012700; and a nucleotide sequence of the gene GmZFP7 is as shown in SEQ ID NO.1.

In some other examples, the gene GmZFP7 regulates isoflavones by activating an expression of a gene GmIFS2 encoding isoflavone synthase 2, and/or inhibiting an expression of a gene GmF3H1 encoding flavanone-3-hydroxylase 1.

In specific examples, the regulating isoflavones refers to increasing or reducing an isoflavone content in a plant; and more specifically, regulating isoflavones refers to increasing or decreasing the content of isoflavones in roots, leaves and seeds of the plant.

Preferably, the plant is selected from the group consisting of soybeans and tobaccos.

Third Group of Examples: Method for regulating isoflavones provided by the present disclosure

The examples in this group provide a method for regulating isoflavones. All the examples in this group have the following common features: the isoflavones are regulated by regulating a level of a soybean C2H2 type zinc finger protein transcription factor GmZFP7 with an amino acid sequence as shown in SEQ ID NO.2, and/or by regulating an expression of a gene GmZFP7 with a gene accession number of Gene ID: 100792169.

In a further example, the regulation of the level of the soybean C2H2 type zinc finger protein transcription factor GmZFP7 with the amino acid sequence as shown in SEQ ID NO.2, and/or the regulation of the expression of the gene GmZFP7 with the gene accession number of Gene ID: 100792169 are/is achieved by way of overexpression, silencing, knockdown or knockout of the gene GmZFP7 encoding the soybean C2H2 type zinc finger protein transcription factor GmZFP7.

In a specific example, the overexpression refers to introducing a recombinant overexpression vector PTF101-GmZFP7-GFP or pGGP-GmZFP7 into a plant, wherein the recombinant overexpression vector is obtained by cloning the gene GmZFP7 into an expression vector PTF101-GFP or pGGP.

Preferably, nucleotide sequences of primers used for cloning the gene GmZFP7 into the expression vectors are as shown in SEQ ID NO.5-6.

The silencing refers to introducing a recombinant silencing expression vector pGGP-GmZFP7-RNAi into a plant, wherein the recombinant silencing expression vector is obtained by cloning an RNAi sequence targeting the gene GmZFP7 into the vector pGGP.

Preferably, nucleotide sequences of primers used for cloning the RNAi sequence targeting the gene GmZFP7 into the vector pGGP are as shown in SEQ ID NO.7-10.

The knockout refers to introducing a JRH0645-GmZFP7 gene editing vector into a plant.

Preferably, the JRH0645-GmZFP7 gene editing vector is constructed by using PCR to implement a series connection of a U6 promoter, gRNA targeting GmZFP7 and gRNA scaffold and then inserting same at an XbaI cleavage site in an original vector JRH0645 (CaMV35s: Cas9).

Preferably, a target sequence of the gRNA is as shown in SEQ ID NO.31.

Experimental Examples: Verification of GmZFP7 protein and gene thereof in regulating isoflavone contents

### I. Materials and Methods

### 1. Experimental Materials

Soybean varieties: Lu Heidou No. 2 (LHD), Williams82
Nicotiana benthamiana
K599 Agrobacterium competent cells (purchased from Zhuangmeng Biotechnology Co., Ltd.)
Escherichia coli DH5 α (purchased from Zhuangmeng Biotechnology Co., Ltd.)
Trans-T1 E. coli competent cells (purchased from TransGen Biotech Co., Ltd.)
EHA105 Agrobacterium competent cells (purchased from Zhuangmeng Biotechnology Co., Ltd.)
ENA105 (psoup) competent cells (purchased from Zhuangmeng Biotechnology Co., Ltd.)
KOD FX High-fidelity enzyme
BAR Quick test strips
18% Glufosinate (Bayer, Germany)
B5 medium basal salt and corresponding vitamins as well as MS medium basal salt and corresponding vitamins purchased from Beijing Ximeijie Technology Co., Ltd., disposable sterile plastic dishes purchased from Beijing Boyuan Hongda Biotechnology Co., Ltd., and plant genome DNA rapid extraction kits (Tiangen Biotechnology Co., Ltd.)
Plant RNA extraction kits purchased from Jinbaite Biotechnology Co., Ltd.
Reverse transcription kits purchased from TransGen Biotech Co., Ltd.
AS (acetosyringone), MES, MgCl₂
Protein rapid extraction kits (Kangwei Biotechnology Co., Ltd.)
Dual-Glo Luciferase Assay System Kit

### 2. Main Instruments and Equipment

PCR amplification instrument (Bio-RAD), electrophoresis instrument (DYY-6C), desktop constant temperature oscillator (THZ-D), high speed freezing centrifuge (SiGMR), high speed tabletop centrifuge (SIGMR 3-30K), gel imaging analyzer (Tanon 3500), thermostatic incubator, etc. (LRH-250A), handheld fluorescence spectrophotometer and Bio-Rad MyiQ monochromatic fluorescence real-time quantitative PCR instrument, LUYOR-3260RB flashlight fluorescent protein observation mirror and Bio-Rad MyiQ monochromatic fluorescence real-time quantitative PCR instrument, and SYNERGY H1 full-featured microplate reader.

### 3. Experimental Methods

### 1) Gene Cloning

RNA was extracted from the first true leaf of LHD, and the RNA of the leaf was reverse-transcribed into cDNA using a reverse transcription kit. The complete coding regions (CDs) of a transcription factor GmZF1 were cloned using the cDNA as a template and GmZF-F/R as primers for subsequent vector construction. At the same time, a plant genome DNA extraction kit was used to extract the genomic DNA of the LHD according to the instructions, and a promoter region at 1500 bp-2000 bp upstream of genes IFS2 (Glyma.13G173500) and F3H1 (Glyma.02G048400) was cloned using the extracted genomic DNA as a template.

### 2) Vector Construction

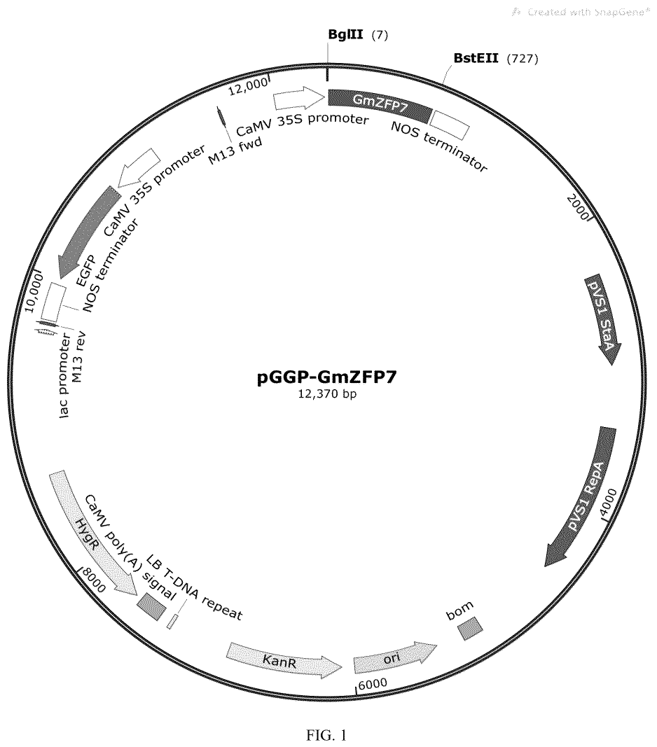

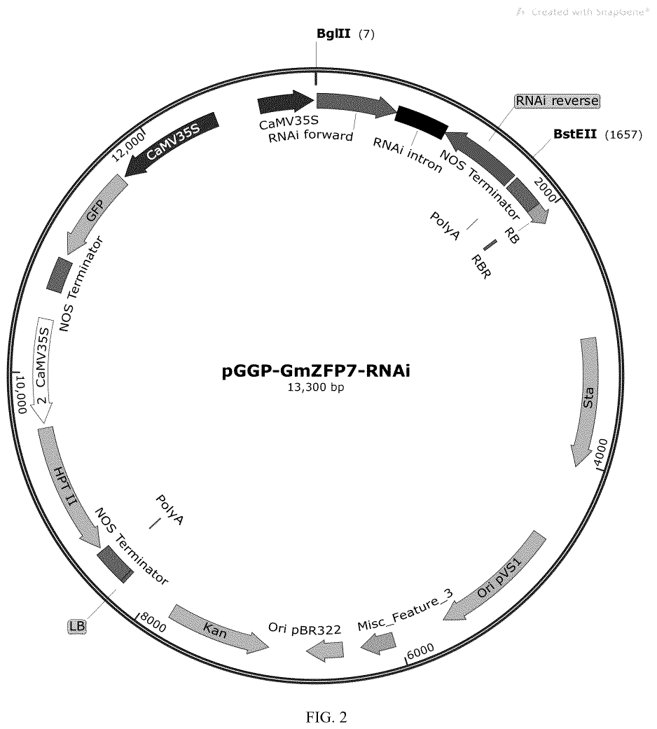

A pGFPGUSplus (pGGP) plant expression vector was used as an original vector in a soybean hairy root experiment, and a GUS gene fragment on the vector was replaced with a desired target gene fragment by performing Bgl II and BstE II double enzyme digestion on the vector. An overexpression vector pGGP-GmZFP7 (FIG. 1) and an inhibitory expression vector pGGP-GmZFP7-RNAi (FIG. 2) for hairy root transformation were separately constructed.

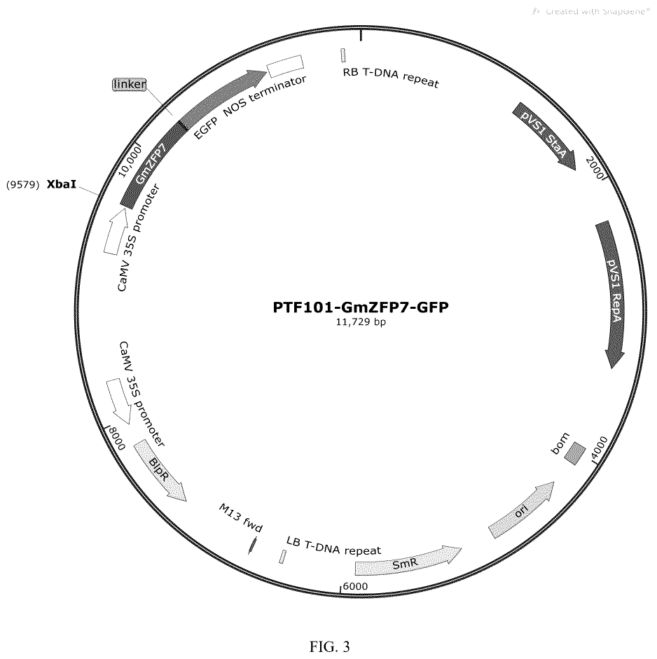

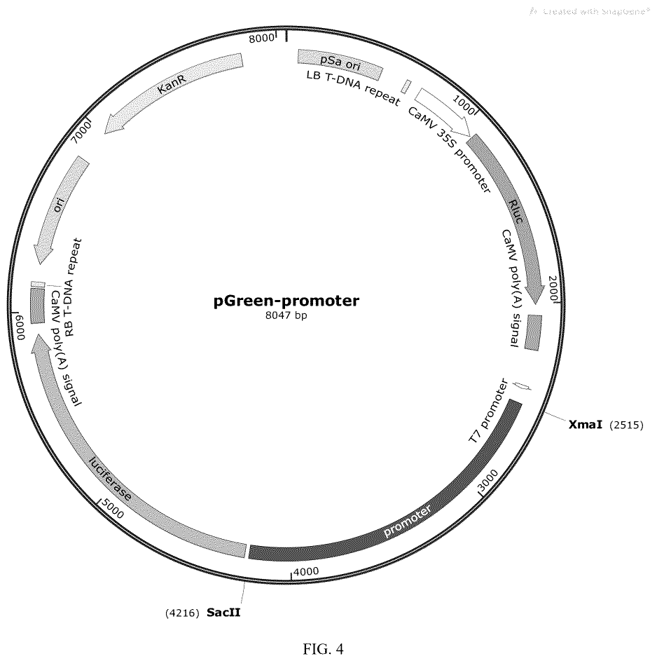

In a dual-luciferase experiment of tobacco, PTF101-GFP and pGreen-080011 were used as original vectors to construct a GmZFP7 plant overexpression vector PTF101-GmZFP7-GFP (FIG. 3) and a signal vector pGmIFS2/pGmF3H1-LUC (FIG. 4).

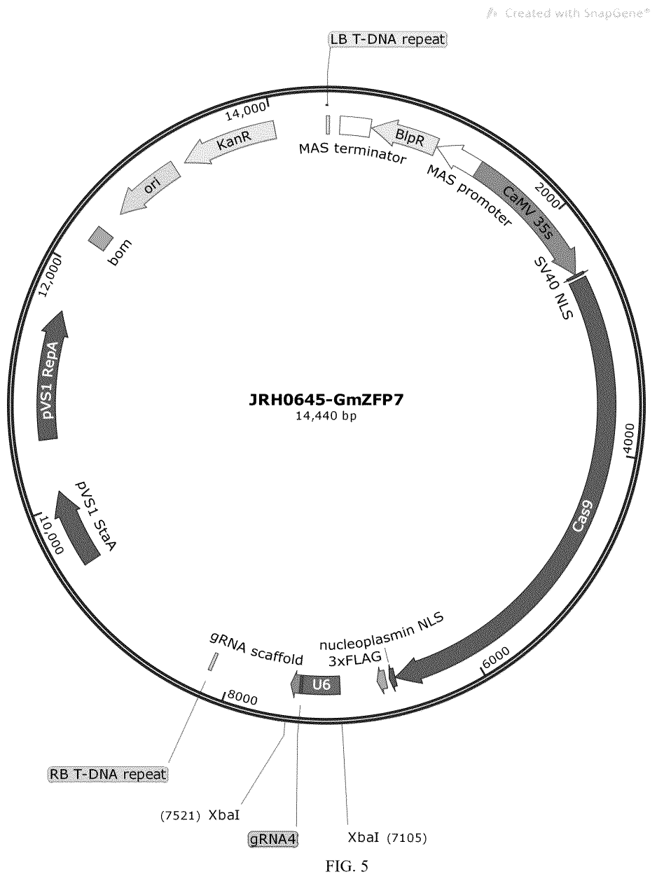

A JRH0645-GmZFP7 gene editing vector (FIG. 5) was constructed by using PCR to implement a series connection of a U6 promoter, gRNA targeting GmZFP7 and gRNA scaffold and then inserting same at the XbaI cleavage site in JRH0645 (CaMV35s: Cas9) which is employed as an original vector of the soybean gene editing vector. The U6 promoter and gRNA scaffold were existing element sequences in the original vector JRH0645 (CaMV35s: Cas9).

See Table 1 for relevant primers.

TABLE 1

| | | Sequence ID |
|---|---|---|
| Primer name | Primer sequence | number |
| GmZFP7-F | ATGATGACTCCAAACTTGAA | SEQ ID No. 3 |
| GmZFP7-R | AAGCCTCAGAGTGAGATCAG | SEQ ID No. 4 |
| GmZFP7-OE-F | ACTCTTGACCATGGTAGATCTATGATGACTCCAAACTTGAA | SEQ ID No. 5 |
| GmZFP7-OE-R | GGGAAATTCGAGCTGGTCACAAGCCTCAGAGTGAGATCAG | SEQ ID No. 6 |
| GmZFP7-RNAi-1-1F | ACTCTTGACCAGATCT CTCTTTTCAGTCGCTCGGACTTGAA | SEQ ID No. 7 |
| GmZFP7-RNAi-1-1R | AGTCATATTAAGCTG GGAGATGCAGATGTTTGTGCTG | SEQ ID No. 8 |
| GmZFP7-RNAi-1-2R | AATTCGAGCT GGTCACC CTCTTTTCAGTCGCTCGGACTTGAA | SEQ ID No. 9 |
| GmZFP7-RNAi-1-2F | ATGAGGTTGCTTCTG GGAGATGCAGATGTTTGTGCTG | SEQ ID No. 10 |
| Intron-F | CAGCTTAATATGACTCTCAA | SEQ ID No. 11 |
| Intron-R | CAGAAGCAACCTCATGGAAA | SEQ ID No. 12 |
| PTF101-ZFP7-F | CACGGGGGACTCTAGAATGATGACTCCAAACTTGAA | SEQ ID No. 13 |
| PTF101-ZFP7-R | GGGGAAATTCGAGCTCTTAAAGCCTCAGAGTGAG | SEQ ID No. 14 |
| PG-IFS2-F | TTCCTGCAGCCCGGGGGATATTCTAAATTCTCCTACT | SEQ ID No. 15 |
| PG-IFS2-R | CGATCTCCACCGCGGCGTGTTCTCGTCCTTGGTTTG | SEQ ID No. 16 |
| PG-F3H1-F | TTCCTGCAGCCCGGGCAGTTCACAGTTTGGAAGCGT | SEQ ID No. 17 |
| PG-F3H-R | CGATCTCCACCGCGGTGTTGTCTTTGAGGAGAATGTGCG | SEQ ID No. 18 |
| U6-F | CTTAGGCCTTCTAGAAAAATAAATGGTAAAATGTC | SEQ ID No. 19 |
| U6-R | AATCCATGTGGTGGCACAT | SEQ ID No. 20 |
| sgRNA-R | AAAAAAAAGCACCGACTCGGGCAACGCGTTCTAGA | SEQ ID No. 21 |
| GmZFP7-gRNA-F | TGTGCCACCACATGGATTGTCTGGTTCCAGATTCAAGTTGTTTTAGAGCTAGA | SEQ ID No. 22 |
| | AATAGC | |

### 3) Transformation of K599 Agrobacterium Competent Cells

The constructed plasmids were added into 100 L of freshly frozen-thawed K599 competent cells, gently mixed, and transferred into an electric shock cup for electric shock transformation. 500 μL of antibiotic-free YEP liquid medium was added, the mixture was blown for 2-3 times with a pipette for mixing well, and the mixed solution was transferred into a 1.5 mL centrifuge tube and shaken on a shaker at 200 rpm and 28° C. for 3 h. An appropriate amount of bacterial solution was taken and then applied onto a YEP solid plate containing corresponding antibiotics, which was incubated in a thermostatic incubator at 28° C. for 36 h-48 h.

### 4) Hairy Root Induction Culture

(a) Seed sterilization: healthy seeds were selected and put into a culture dish, and a chlorine sterilization method was adopted (80 mL of sodium hypochlorite and 5 mL of concentrated hydrochloric acid were added into a beaker). The culture dish and soybean seeds were put together in a dryer for airtight sterilization for about 16 h-18 h.
(b) Seed germination: the above sterilized seeds were planted in a germination medium, and germinated at 25° C. with 16 hours of light/8 hours of darkness every day.
(c) Preparation of bacterial solution: the preserved bacterial solution was taken and activated twice in a YEP liquid medium, and the activated bacterial solution was incubated at 28° C. on a constant temperature shaker at 200 rpm until the OD₆₀₀ of the culture was about 0.6-0.8.
(d) Obtaining explants: soybean seeds that had germinated for 4-7 days were taken and then cut off at 1 mm-2 mm away from the hypocotyl, each of the cotyledons was split in half, and the apical buds were removed. 5-7 wounds were gently scratched at the cotyledonary nodes with a blade, so that explants were obtained.
(e) Explant infection: the bacterial solution was centrifuged at 6000 rpm for 10 min, a liquid co-medium was used to resuspend the cells and the OD₆₀₀ of the suspension was adjusted to 0.6-0.8. The cotyledons were infected using the suspension for 15 min-20 min.
(f) Co-cultivation: the cotyledons were transferred to a solid co-medium covered with sterile filter paper, and cultured in darkness for 3 days at 25° C.
(g) Hairy root induction culture: the explants obtained after co-cultivation were washed with double distilled water containing antibiotics for 3-5 times, then transferred into a hairy root induction medium, and cultured at 25° C. with 16 hours of light/8 hours of darkness every day.
(h) Detection of hairy roots: the induced culture dish was irradiated with a fluorescent microscope, positive roots showed green fluorescence in the field of view, and the positive roots and negative roots were counted and sampled.
   5) EHA105 and EHA105 (pSoup) Agrobacterium Competent Cell Transformation and Tobacco Transient Transformation

100 μL of competent cells were taken and added with plasmid DNA, which were mixed well; and the obtained mixture was placed on ice for 5 min, in liquid nitrogen for 5 min, in water bath at 37° C. for 5 min and in ice bath for 5 min in turn for heat shock transformation. An LB liquid medium without antibiotics was added, and the cells were incubated at 28° C. on a shaker for 2 h-3 h. The culture was centrifuged at 5000 rpm for 1 min to collect bacteria, 100 μL of supernatant was left and gently blown to resuspend bacterial blocks, the suspension was applied onto an LB plate containing the corresponding antibiotic, and the LB plate was inverted and incubated in an incubator at 28° C. for 2-3 days.

Newly activated Agrobacterium monoclonal was inoculated into YEP containing the corresponding antibiotic and incubated overnight at 28° C. and 200 rpm. When the OD₆₀₀ of the bacteria solution was within a range of 0.6-1.0, cells were collected by centrifugation at 1000 g for 5 min. The collected cells were gently resuspended with 2 mL of Induction medium, and then cells were collected by centrifugation again. The resulting precipitate was resuspended with 1 mL of Induction medium. The suspension was placed at room temperature for 1 h-4 h to measure the OD value, and an infection solution was prepared according to the needs of the experiment. The infection solution was injected into the leaves of tobaccos (Nicotiana benthamiana) grown for 6-8 weeks using a syringe, and then the tobaccos were cultured in an incubator for 48 h-72 h after dark treatment for 12 h.

### 6) Detection of Fluorescence Signals Luc and Ren in Tobacco Leaves

After 48 h to 72 h of tobacco injection, whether there were GFP fluorescence signals at the injection sites of the leaves was observed by using a fluorescence protein observation microscope. 0.5 g of tobacco leaves with strong GFP signals were taken and ground with liquid nitrogen. Cytoplasmic soluble protein was extracted from the leaves and then placed on ice for later use. A Dual-Glo Luciferase Assay System kit stored at −20° C. was taken out and placed on ice for thawing. Dual-Glo Luciferase Substrate and Dual-Glo Luciferase Buffer in the kit were mixed together (called Buffer-Luc) and placed on ice for later use. Dual-Glo Stop & Glo Buffer and Dual-Glo Stop & Glo Substrate were mixed together (called Buffer-Ren) and placed on ice for later use. The cytoplasmic protein extracted from tobacco and the Buffer-Luc were added into an ELISA plate and mixed well by blowing with a pipette, and then the activity of Luc was detected with a SYNERGY HI full-featured microplate reader. After Luc detection was completed, 75 μL of Buffer-Ren was added into the above mixed system and mixed well by blowing so as to terminate the Luc reaction, and the Ren reaction was then started for detection.

### 7) Genetic Transformation of Soybeans

Seed sterilization: the soybean variety Williams82 was selected, and the seeds were sterilized by using a chlorine sterilization method. After that, a culture dish with sterilized seeds was placed on an ultra-clean bench and blown for 15 min to remove residual chlorine.

Seed germination: the sterilized soybean seeds were taken and planted in a germination medium with the umbilicus facing down. After sealing, incubation was performed at 25° C. for 16 h, with 16 hours of light/8 hours of darkness every day.

Preparation of bacterial solution: 40 μL-60 μL of Agrobacterium bacterial solution was evenly applied to a solid medium of YEP containing Rifampicin and the antibiotic corresponding to the vector, and incubated upside down in a dark incubator at 28° C. for 48 h; after activation, the cells were resuspended with double distilled water, and then reapplied evenly on a medium containing corresponding antibiotics; after sealing, the medium was inverted in a dark incubator at 28° C. for 24 h; and then the cells were resuspended with a resuspension solution until the OD₆₀₀ of the bacterial solution was 0.6-0.8. The bacterial solution was used for subsequent infection.

Preparation of cotyledonary node explants: the seed coat was removed, every two cotyledons were separated from each other, the cotyledons were gently removed with a knife, and a few light cuts were made at the junctions of the cotyledonary nodes. The treated seeds were put into the bacterial solution and infected for 1.5 h.

Co-cultivation: the prepared cotyledonary node explants were put into the prepared bacterial solution and soaked for 1.5 h, and the soaked cotyledonary node explants were placed with the convex sides down on a CCM medium covered with sterile filter paper. After sealing, the cotyledonary node explants were cultured in the dark at 22° C. for 5 days.

Recovery induction culture: the explants were taken out after being subjected to co-cultivation for 5 days, the explants were washed for 4-5 times with a liquid induction medium without sucrose and agar, then the surface moisture was dried with filter paper, and the explants were inserted into the induction medium at a tilt angle of 45°. After sealing, the explants were placed in a culture room at 25° C. for 7 days, with 16 hours of light/8 hours of darkness every day.

Screening induction culture: the soybean explants were taken after being subjected to the recovery culture, the excessively long hypocotyl and cluster buds were cut off, part of the radicle was cut off, with about 0.5 cm radicle being reserved, and the explants were inserted into a screening medium at a tilt angle of 45°. After sealing, the explants were placed in a culture room at 25° C. for 21 days, with 16 hours of light/8 hours of darkness every day.

Elongation subculture: the explants were taken after being subjected to screening culture for 21 days, part of the hypocotyl was cut off, and the explants were inserted into an elongation medium at a tilt angle of 45°. After sealing, the explants were cultured at 25° C., with 16 hours of light/8 hours of darkness every day. The first elongation culture lasted for 21 days, and subsequent elongation subcultures might be performed once every 15 days, with 2-3 repetitions. The transgenic seedlings after elongation subculture were washed and transplanted.

### 8) Bar Identification of Transgenic Plants

The screening was mainly carried out using two methods: BAR test strips and glufosinate spraying or application.

BAR rapid test strip: a small amount of leaves were taken and put into a 1.5 mL centrifuge tube, a small amount of water was added to break the leaf tissue, and a BAR test strip was inserted into the leaf fragments for about 3 min. When a stripe appeared at the end of the test strip far away from liquid surface, that was, the test strip was completely wetted by the liquid, the test results could be observed.

Glufosinate spraying or application: 18% glufosinate was diluted 1000 times and then applied onto the surfaces of the leaves, and the leaves were marked; and the test results could be observed 3-5 days after the application.

### 9) PCR Sequencing Identification of Gene-Edited Plants

For gene-edited plants, in addition to the related detection of the Bar gene, PCR amplification and sequencing of the CRISPR-Cas9 gene were mainly used for detection. The leaves of the transgenic plants at the flowering stage were taken, and the whole genome DNA in the leaves was extracted using the Plant Whole Genome DNA Extraction Kit produced by Tiangen Company. Cas9 detection and downstream editing gene detection were performed on CRISPR gene edited plants. PCR primers are shown in Table 2.

TABLE 2
Primers for detection of CRISPR edited plants

| Primer name | Primer sequence | Sequence ID number |
|---|---|---|
| Cas9-test-F | GACAAGAAGTATAGTATTGGTCTGG | SEQ ID No. 23 |
| Cas9-test-R | CCTCTGTGCCATCCATCTTCTC | SEQ ID No. 24 |
| CR-ZF20-F | TAAAAGGAAGTGGAGTTACATTG | SEQ ID No. 25 |
| CR-ZF20-R | TTAAAGCCTCAGAGTGAGATCAG | SEQ ID No. 26 |

### 10) Extraction and Detection Methods of Isoflavones

A cyclone mill (mortar) was used to grind the tissue to be tested into powder, 0.02 g of powder was weighed and put into a 2 mL centrifuge tube, 1 mL of an extraction solution containing 70% (v/v) ethanol and 0.1 (v/v) acetic acid was added, and then the obtained mixture was shaken and mixed for 12 h on a shaker. The completely mixed solution was centrifuged at 4° C., 2700 g for 10 min, and the supernatant was collected. The supernatant was filtered using a filter (YMC, Kyoto, Japan) having a pore size of 0.2 m. Isoflavone content was determined using an Agilent 1260 HPLC system (Agilent Technologies, Santa Clara, California, USA). A YMC ODS AM-303 chromatographic column (250 mm×4.6 mm I.D., S-5 μm, 120 Å) was used for quantitative analysis. Mobile phases A and B were composed of 0.1% acetic acid and acetonitrile dissolved in distilled water, respectively. The solvent flow rate was 1.0 mL·min⁻¹, the injection volume was 10 μL, and a 70-minute linear gradient of 13%-30% acetonitrile (v/v) was used. The wavelength of an ultraviolet detector was set to 260 nm, and the column temperature was set to 35° C. Standard samples of soybean isoflavones were composed of 12 components including genistin, daidzin, glycitin, malonyl genistin, malonyl daidzin, malonyl glycitin, acetyl daidzin, acetyl genistin, acetyl glycitin, genistein, daidzein, and glycitein. All the standard samples with concentrations of 200 g mL⁻¹ were mixed together in equal amounts to prepare a mixed standard sample, and the prepared mixed standard sample was placed at −20° C. for later use. Qualitative analysis was conducted based on the retention time and maximum absorption spectra of 12 isoflavone standard samples, and the ultraviolet absorption value at 260 nm was used as the standard. Referring to Sun Junming et al. (2011) method (Sun J, Sun B, Han F, Yan S, Yang H and Kikuchi A. Rapid HPLC method for determination of 12 isoflavone components in soybean seeds. Agri Sci China, 2011, 10(1): 101-105), the components, aglycones and total contents of isoflavones in the samples were calculated.

11) qRT-PCR Analysis

Real-time PCR was used to detect the expression levels of silenced and overexpressed genes in soybean hairy roots. A GmActin-11-like gene was used as a reference gene, and the operation was performed according to instructions of the SYBR Premix Ex taqII of TaKaRa Company. The nucleotide sequences of primers used in the PCR are shown in Table 3. The gene expression levels were calculated by a 2^{-ΔΔCT} method.

TABLE 3

| Primer name | Primer sequence | Sequence ID number |
|---|---|---|
| qGmZFP7-F | ATGATGACTCCAAACTTGAATCTGG | SEQ ID No. 27 |
| qGmZFP7-R | TAACTTTCAACTCTTCATCACCGGG | SEQ ID No. 28 |
| qGmActin-F | CGGTGGTTCTATCTTGGCATC | SEQ ID No. 29 |
| qGmActin-R | GTCTTTCGCTTCAATAACCCTA | SEQ ID No. 30 |

### II. Experimental Results

### 1. Overexpression and Silencing of GmZFP7 Significantly Change the Isoflavone Content in Soybean Hairy Roots.

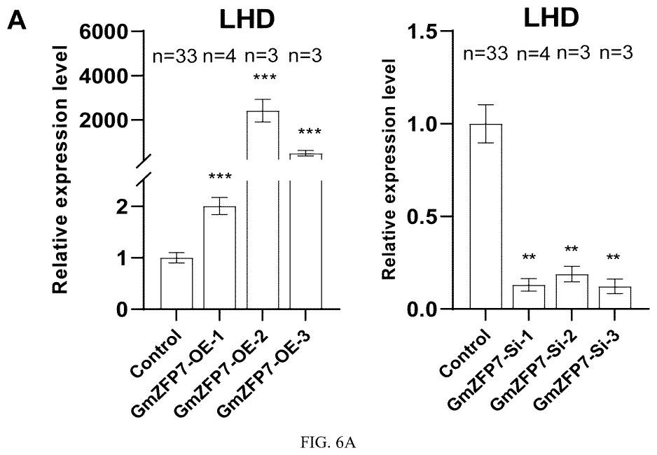

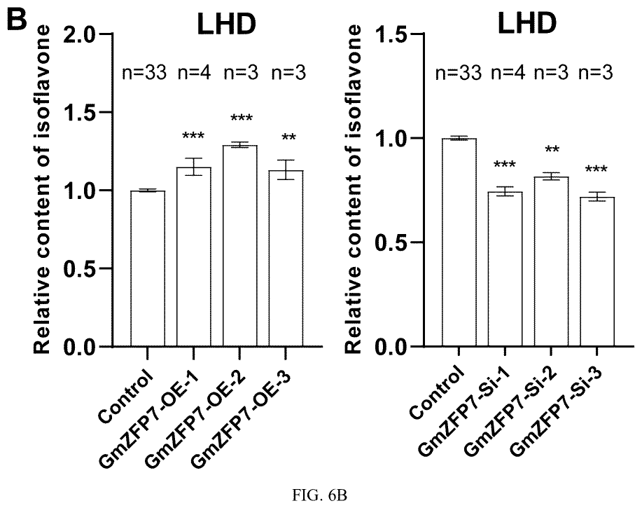

In Lu Heidou No. 2, overexpression of GmZFP7 through the pGGP-GmZFP7 vector resulted in 1-2420 times increase in the relative expression level of the GmZFP7 in transgenic hairy roots and a 13%-29% increase in total flavonoid content. After the GmZFP7 was silenced by the pGGP-GmZFP7-RNAi vector, the relative expression level of the GmZFP7 decreased by 81%-88%, and the total isoflavone content decreased by 18%-28%, indicating that overexpression and silencing of the GmZFP7 significantly changed the total isoflavone content in soybean hairy roots (FIG. 6). 2. GmZFP7 significantly activates the gene expression of node enzyme-isoflavone synthase 2 (GmIFS2) in an isoflavone synthesis pathway, while inhibiting the gene expression of flavanone-3-hydroxylase 1 (GmF3H1), which competes with it for common substrates.

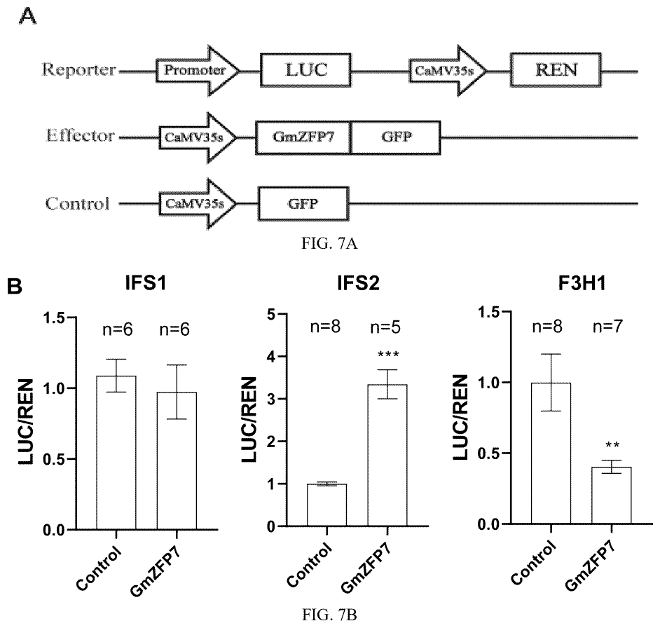

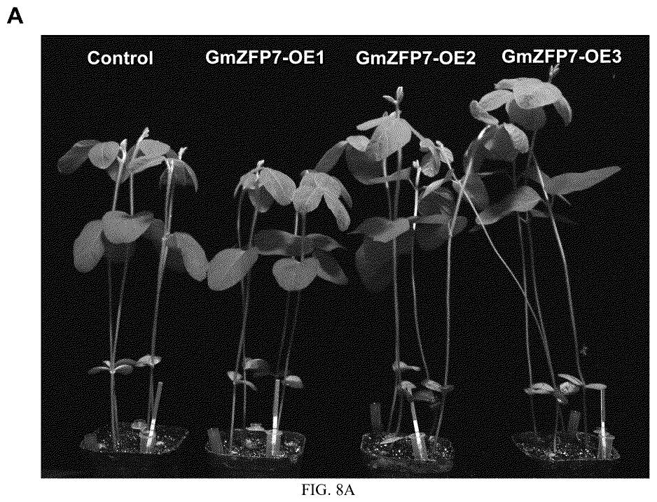

CaMV35s:Ren was used as a reference, and the PTF101-GFP vector was used as a control to conduct experiments. The regulation of the transcription factor GmZFP7 on the GmIFS1, GmIFS2 and GmF3H1 promoters was determined by detecting the Luc enzyme activity in the pGreen vector. The results showed that after the GmZFP7 was expressed, the activation activity of the GmIFS1 promoter did not change significantly, the activation activity of the GmIFS2 promoter was increased by 3.3 times, and the activation activity of the GmF3H1 promoter was reduced by 60% (FIG. 7). The results indicated that GmZFP7 increased isoflavone content by activating the gene expression of key node enzyme-isoflavone synthase 2 (GmIFS2) in an isoflavone synthesis pathway, while inhibiting the gene expression of flavanone-3-hydroxylase 1 (GmF3H1), which competes with it for common substrates.

3. The Phenotype of Total Isoflavones in Soybean Stably Transformed Plants Indicates that the Contents of Isoflavones in Soybean Leaves and Seeds can be Significantly Changed by Modifying GmZFP7.
3.1 Obtaining and Identification of Plants with GmZFP7 Overexpressed

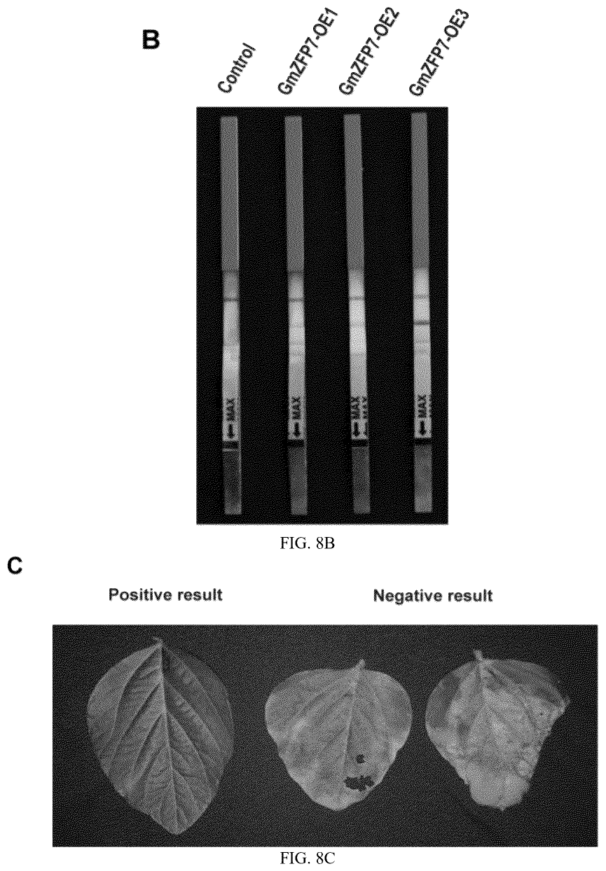

Through the soybean cotyledonary node genetic transformation method, the GmZFP7 overexpression vector PTF101-GmZFP7-GFP and the control vector PTF101-GFP were transferred into the soybean variety Williams82 (FIG. 8). The obtained overexpression lines were detected by Bar test strips, glufosinate application screening, and qPCR detection of GmZFP7. A total of three GmZFP7 overexpression homozygous lines in T4 generation with Bar genes and glufosinate-resistance were screened out, and three lines with empty vectors PTF101-GFP were used as controls. The detection results are shown in FIG. 8.

### 3.2 Overexpression of GmZFP7 can Increase the Content of Isoflavones in Soybean Leaves and Seeds.

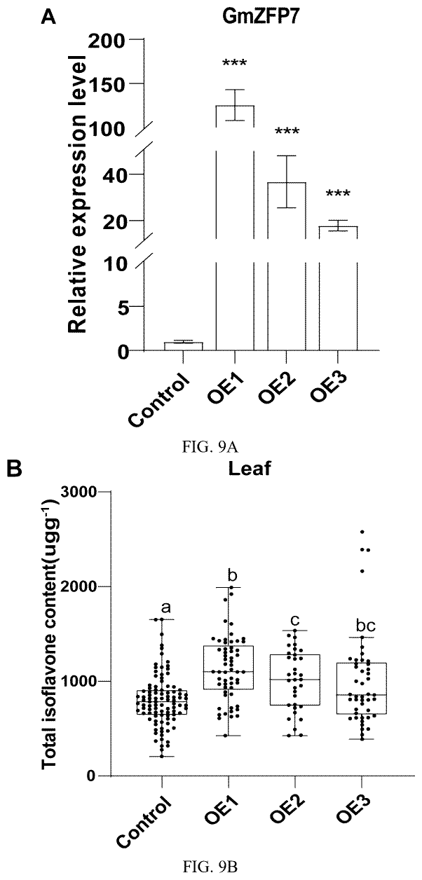

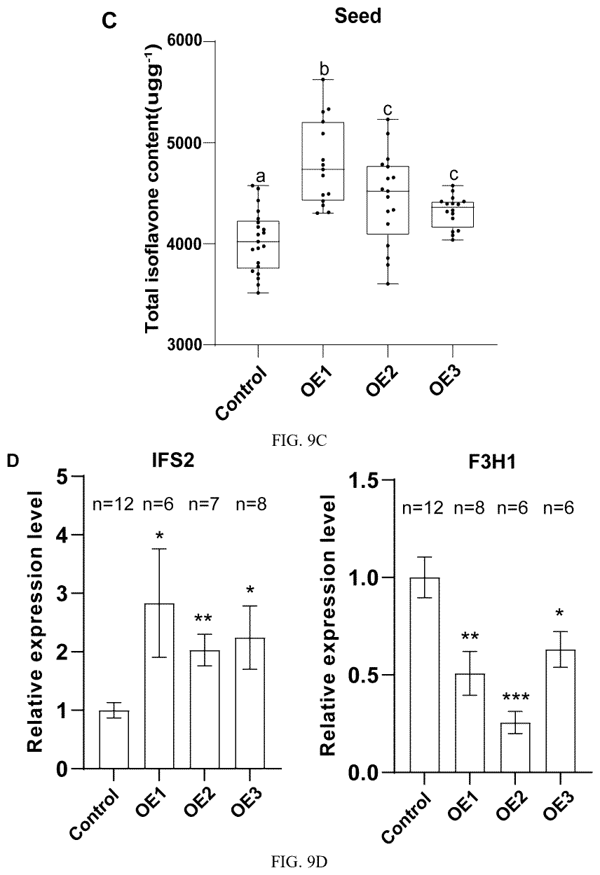

The leaves and seeds of the stably genetic transgenic plants in T4 generation were selected to detect the gene expression level of GmZFP7 and the total isoflavone content. In the three GmZFP7 overexpression lines OE1, OE2 and OE3, the relative expression levels of the GmZFP7 increased by 17-125 times compared with the control (FIG. 9A). The significant difference in GmZFP7 gene expression levels among the three overexpression lines may be caused by the different copy numbers of GmZFP7 inserted in the genome. However, compared with the control line, all the three overexpression lines showed a significant increase in expression levels.

Moreover, the contents of total isoflavones in leaves and seeds were significantly increased, where the content of total isoflavones in the leaves was increased by 35%-39%, and the content of total isoflavones in the seeds was significantly increased by 7%-19% (FIG. 9B and FIG. 9C). On the basis of transient expression experiments in hairy roots and tobaccos, we detected the changes in the relative expression levels of GmIFS2 and GmF3H1 in transgenic plants. In the leaves of the three overexpression lines, the relative expression level of GmIFS2 increased by 2.0-2.8 times, while the expression level of GmF3H1 decreased by 40%-80% (FIG. 9D). The above results indicated that GmZFP7 could regulate the metabolic direction of a phenylpropane metabolic pathway by increasing the expression of GmIFS2 in the isoflavone pathway and inhibiting the expression of GmF3H1 in the flavonol pathway so as to increase the isoflavone content. 3.3 Screening of plants with GmZFP7 knockout mediated by CRISPR/Cas9

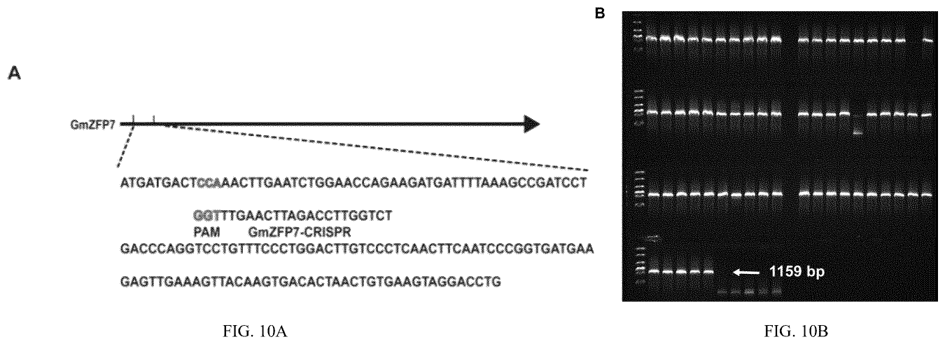

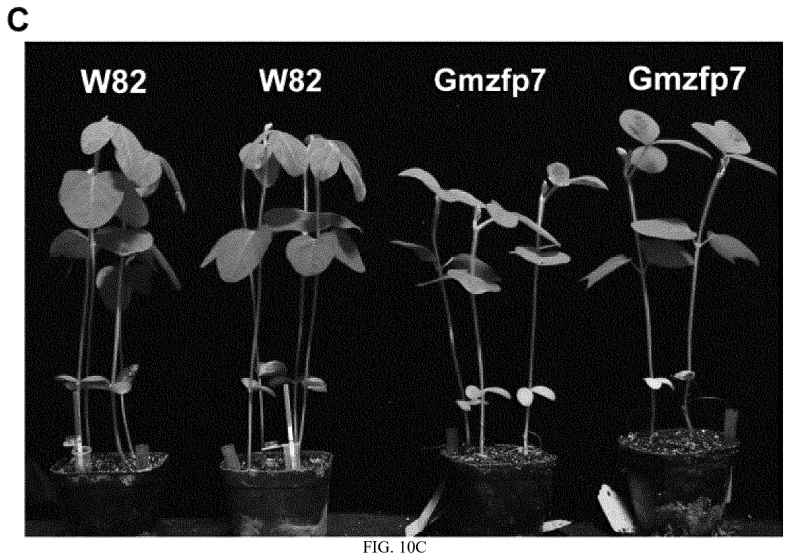

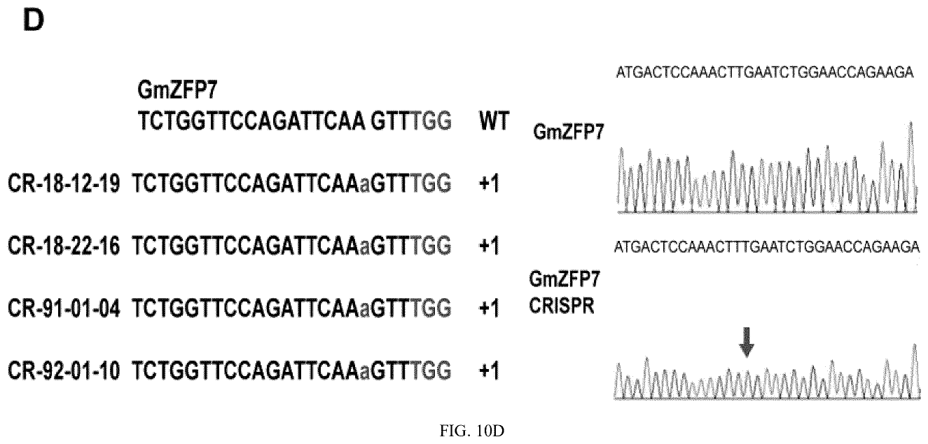

According to the sequence features of GmZFP7, a suitable sgRNA target sequence GGTTTGAACTTAGACCTTGGTCT (SEQ ID NO.31) was designed and screened out online using CRISPR-P2.0 (FIG. 10A) (PAM is shown in red font). For the TO-T1 generations of transgenic edited plants, Bar gene screening and Cas9 gene molecular identification were first carried out, and individual plants were sown and harvested (FIG. 10B). 31 bimodal heterozygous edited materials were screened from gene edited materials of three lines in T2 generation, including 4 homozygous mutants with the same editing type, all of which were single base insertions (FIG. 10C and FIG. 10D).

### 3.4 GmZFP7 Knockout Significantly Reduces Isoflavone Content in Leaves and Seeds of Transgenic Plants

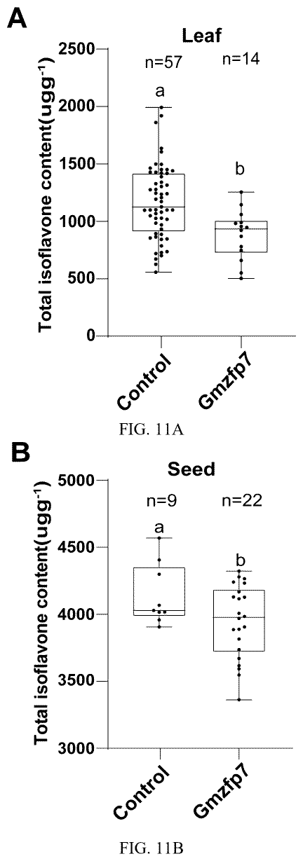

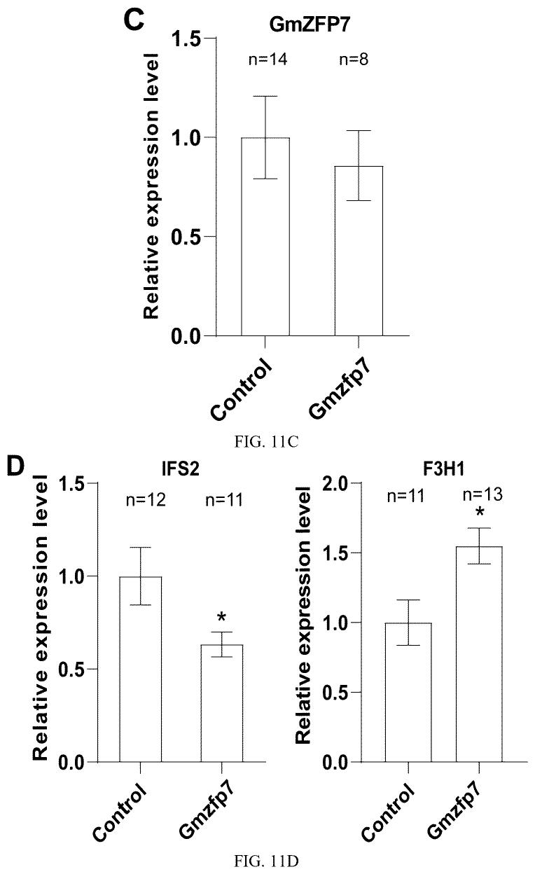

The homozygous knockout mutant Gmzfp7 plants screened from the T2 generation transgenic plants with positive results were harvested individually. T3 generation plants were selected, and the total isoflavone contents and relative expression levels of related genes in the leaves and seeds of the T3 generation plants were detected. The total isoflavone contents in the leaves and seeds of the Gmzfp7 mutant were significantly reduced (FIG. 11A and FIG. 11B). The results of qPCR detection showed that the expression level of GmZFP7 in the mutant did not change significantly, the expression level of GmIFS2 was significantly decreased, and the expression level of GmF3H1 was significantly increased, which were consistent with the above results, indicating that GmZFP7 could change the phenylpropane metabolic direction by increasing the expression of GmIFS2 in the isoflavone pathway and inhibiting the expression of GmF3H1 in the flavonol pathway so as to increase the total isoflavone content.

What is claimed is:

1. A method for regulating isoflavones, comprising using soybean C2H2 type zinc finger protein transcription factor GmZFP7 with the amino acid sequence as shown in SEQ ID NO: 2.

2. The method for regulating the isoflavones according to claim 1, wherein a gene accession number of a gene GmZFP7 encoding the soybean C2H2 type zinc finger protein transcription factor GmZFP7 is Gene ID: 100792169.

3. The method for regulating the isoflavones according to claim 2, wherein the nucleotide sequence of the gene GmZFP7 encoding the soybean C2H2 type zinc finger protein transcription factor GmZFP7 is as shown in SEQ ID NO: 1; and/or
the method comprises increasing or reducing an isoflavone content in a plant by increasing or decreasing a level of the soybean C2H2 type zinc finger protein transcription factor GmZFP7 in a plant body, or by an overexpression, a silencing, a knockdown, or a knockout of the gene GmZFP7 encoding the soybean C2H2 type zinc finger protein transcription factor GmZFP7 in the plant body; and/or
the plant is selected from the group consisting of soybeans and tobaccos.

4. A method for regulating isoflavones, comprising using a gene GmZFP7 with a gene accession number of Gene ID: 100792169.

5. The method for regulating the isoflavones according to claim 4, wherein a gene locus number of the gene GmZFP7 is Glyma.20G012700; and the nucleotide sequence of the gene GmZFP7 is as shown in SEQ ID NO: 1.

6. The method for regulating the isoflavones according to claim 4, wherein the gene GmZFP7 regulates the isoflavones by activating an expression of a gene GmIFS2 encoding isoflavone synthase 2, and/or inhibiting an expression of a gene GmF3H1 encoding flavanone-3-hydroxylase 1.

7. The method for regulating the isoflavones according to claim 4, wherein the method comprises increasing or reducing an isoflavone content in a plant; and/or
the plant is selected from the group consisting of soybeans and tobaccos.

8. A method for regulating isoflavones, comprising: regulating a level of a soybean C2H2 type zinc finger protein transcription factor GmZFP7 with the amino acid sequence as shown in SEQ ID NO: 2, and/or regulating an expression of a gene GmZFP7 with a gene accession number of Gene ID: 100792169.

9. The method for regulating the isoflavones according to claim 8, wherein a regulation of the level of the soybean C2H2 type zinc finger protein transcription factor GmZFP7 with the amino acid sequence as shown in SEQ ID NO: 2, and/or a regulation of the expression of the gene GmZFP7 with the gene accession number of Gene ID: 100792169 are/is achieved by an overexpression, a silencing, a knockdown, or a knockout of the gene GmZFP7 encoding the soybean C2H2 type zinc finger protein transcription factor GmZFP7.

10. The method for regulating the isoflavones according to claim 9, wherein the overexpression comprises introducing a recombinant overexpression vector PTF101-GmZFP7-GFP or pGGP-GmZFP7 into a first plant, wherein the recombinant overexpression vector PTF101-GmZFP7-GFP or pGGP-GmZFP7 is obtained by cloning the gene GmZFP7 into an expression vector PTF101-GFP or pGGP; and/or
the nucleotide sequences of primers used for cloning the gene GmZFP7 into the expression vector PTF101-GFP or pGGP are as shown in SEQ ID NOS: 5-6;
the silencing comprises introducing a recombinant silencing expression vector pGGP-GmZFP7-RNAi into a second plant, wherein the recombinant silencing expression vector pGGP-GmZFP7-RNAi is obtained by cloning an RNAi sequence targeting the gene GmZFP7 into the expression vector pGGP; and/or
the nucleotide sequences of primers used for cloning the RNAi sequence targeting the gene GmZFP7 into the expression vector pGGP are as shown in SEQ ID NOS: 7-10;
the knockout comprises introducing a JRH0645-GmZFP7 gene editing vector into a third plant; and/or
the JRH0645-GmZFP7 gene editing vector is constructed by using a polymerase chain reaction (PCR) to implement a series connection of a U6 promoter, a gRNA targeting the gene GmZFP7, and a gRNA scaffold, and inserting a fragment formed by the series connection of the U6 promoter, the gRNA targeting the GmZFP7, and the gRNA scaffold at an XbaI cleavage site in an original vector JRH0645 (CaMV35s: Cas9); and/or
a target sequence of the gRNA targeting the gene GmZFP7 is as shown in SEQ ID NO: 31.

11. The method for regulating the isoflavones according to claim 5, wherein the gene GmZFP7 regulates the isoflavones by activating an expression of a gene GmIFS2 encoding isoflavone synthase 2, and/or inhibiting an expression of a gene GmF3H1 encoding flavanone-3-hydroxylase 1.

12. The method for regulating the isoflavones according to claim 5, wherein the method comprises increasing or reducing an isoflavone content in a plant; and/or
the plant is selected from the group consisting of soybeans and tobaccos.


SEQUENCE LISTING (36 sequences)

SEQ ID NO: 1 - DNA, 717 bp, synthetic construct
  misc_feature 1..717: note = C2H2GmZFP7
  source 1..717: mol_type = other DNA
atgatgactccaaacttgaatctggaaccagaagatgattttaaagtcctcatccaagtaggttctgataagtcgatacgagagacatcacatgatcttaccaaggccgatcctgacccaggtcctgtttccctggacttgtccctcaacttcaatcccggtgatgaagagttgaaagttacaagtgacactaactgtgaagtaggacctgaaactcatgcctcagcatcagcaattcctagagttttctcttgtaattattgctggcggaaattctttagctcacaggcattaggtggacaccagaatgctcacaaaagggagaggacaatggcgaagcacgcaatgcgaatggggatgttcgctgaaaggtacacaagcctagcatctctgcctcttcatggttcctcttttcagtcgctcggacttgaagctcatgctgcaatgcaccaaggacatgtgcatcatcattcaatgagggctcctgatataagagctgcagcaaaattcggtaaagactattttcggatgccgatcttcttggaagatgatgatgtcggtctattttggcctggaagtttcagacagactgatgaaaggggtggtgttaatttggggcatggtgagcatgctcataattcaaatacttgttttgtagtagcaactgcacctccagcacaaacatctgcatctcctgatctcactctgaggctttaa

SEQ ID NO: 2 - protein, 238 aa, synthetic construct
  REGION 1..238: note = C2H2GmZFP7
MMTPNLNLEPEDDFKVLIQVGSDKSIRETSHDLTKADPDPGPVSLDLSLNFNPGDEELKVTSDTNCEVGPETHASASAIPRVFSCNYCWRKFFSSQALGGHQNAHKRERTMAKHAMRMGMFAERYTSLASLPLHGSSFQSLGLEAHAAMHQGHVHHHSMRAPDIRAAAKFGKDYFRMPIFLEDDDVGLFWPGSFRQTDERGGVNLGHGEHAHNSNTCFVVATAPPAQTSASPDLTLRL

SEQ ID NO: 3 - DNA, 20 bp, synthetic construct
  misc_feature 1..20: note = GmZFP7-F
  source 1..20: mol_type = other DNA
atgatgactccaaacttgaa

SEQ ID NO: 4 - DNA, 20 bp, synthetic construct
  misc_feature 1..20: note = GmZFP7-R
  source 1..20: mol_type = other DNA
aagcctcagagtgagatcag

SEQ ID NO: 5 - DNA, 41 bp, synthetic construct
  misc_feature 1..41: note = GmZFP7-OE-F
  source 1..41: mol_type = other DNA
actcttgaccatggtagatctatgatgactccaaacttgaa

SEQ ID NO: 6 - DNA, 40 bp, synthetic construct
  misc_feature 1..40: note = GmZFP7-OE-R
  source 1..40: mol_type = other DNA
gggaaattcgagctggtcacaagcctcagagtgagatcag

SEQ ID NO: 7 - DNA, 41 bp, synthetic construct
  misc_feature 1..41: note = GmZFP7-RNAi-1-1F
  source 1..41: mol_type = other DNA
actcttgaccagatctctcttttcagtcgctcggacttgaa

SEQ ID NO: 8 - DNA, 37 bp, synthetic construct
  misc_feature 1..37: note = GmZFP7-RNAi-1-1R
  source 1..37: mol_type = other DNA
agtcatattaagctgggagatgcagatgtttgtgctg

SEQ ID NO: 9 - DNA, 42 bp, synthetic construct
  misc_feature 1..42: note = GmZFP7-RNAi-1-2R
  source 1..42: mol_type = other DNA
aattcgagctggtcaccctcttttcagtcgctcggacttgaa

SEQ ID NO: 10 - DNA, 37 bp, synthetic construct
  misc_feature 1..37: note = GmZFP7-RNAi-1-2F
  source 1..37: mol_type = other DNA
atgaggttgcttctgggagatgcagatgtttgtgctg

SEQ ID NO: 11 - DNA, 20 bp, synthetic construct
  misc_feature 1..20: note = Intron-F
  source 1..20: mol_type = other DNA
cagcttaatatgactctcaa

SEQ ID NO: 12 - DNA, 20 bp, synthetic construct
  misc_feature 1..20: note = Intron-R
  source 1..20: mol_type = other DNA
cagaagcaacctcatggaaa

SEQ ID NO: 13 - DNA, 36 bp, synthetic construct
  misc_feature 1..36: note = PTF101-ZFP7-F
  source 1..36: mol_type = other DNA
cacgggggactctagaatgatgactccaaacttgaa

SEQ ID NO: 14 - DNA, 34 bp, synthetic construct
  misc_feature 1..34: note = PTF101-ZFP7-R
  source 1..34: mol_type = other DNA
ggggaaattcgagctcttaaagcctcagagtgag

SEQ ID NO: 15 - DNA, 37 bp, synthetic construct
  misc_feature 1..37: note = PG-IFS2-F
  source 1..37: mol_type = other DNA
ttcctgcagcccgggggatattctaaattctcctact

SEQ ID NO: 16 - DNA, 36 bp, synthetic construct
  misc_feature 1..36: note = PG-IFS2-R
  source 1..36: mol_type = other DNA
cgatctccaccgcggcgtgttctcgtccttggtttg

SEQ ID NO: 17 - DNA, 36 bp, synthetic construct
  misc_feature 1..36: note = PG-F3H1-F
  source 1..36: mol_type = other DNA
ttcctgcagcccgggcagttcacagtttggaagcgt

SEQ ID NO: 18 - DNA, 39 bp, synthetic construct
  misc_feature 1..39: note = PG-F3H-R
  source 1..39: mol_type = other DNA
cgatctccaccgcggtgttgtctttgaggagaatgtgcg

SEQ ID NO: 19 - DNA, 35 bp, synthetic construct
  misc_feature 1..35: note = U6-F
  source 1..35: mol_type = other DNA
cttaggccttctagaaaaataaatggtaaaatgtc

SEQ ID NO: 20 - DNA, 19 bp, synthetic construct
  misc_feature 1..19: note = U6-R
  source 1..19: mol_type = other DNA
aatccatgtggtggcacat

SEQ ID NO: 21 - DNA, 35 bp, synthetic construct
  misc_feature 1..35: note = sgRNA-R
  source 1..35: mol_type = other DNA
aaaaaaaagcaccgactcgggcaacgcgttctaga

SEQ ID NO: 22 - DNA, 59 bp, synthetic construct
  misc_feature 1..59: note = GmZFP7-gRNA-F
  source 1..59: mol_type = other DNA
tgtgccaccacatggattgtctggttccagattcaagttgttttagagctagaaatagc

SEQ ID NO: 23 - DNA, 25 bp, synthetic construct
  misc_feature 1..25: note = Cas9-test-F
  source 1..25: mol_type = other DNA
gacaagaagtatagtattggtctgg

SEQ ID NO: 24 - DNA, 22 bp, synthetic construct
  misc_feature 1..22: note = Cas9-test-R
  source 1..22: mol_type = other DNA
cctctgtgccatccatcttctc

SEQ ID NO: 25 - DNA, 23 bp, synthetic construct
  misc_feature 1..23: note = CR-ZF20-F
  source 1..23: mol_type = other DNA
taaaaggaagtggagttacattg

SEQ ID NO: 26 - DNA, 23 bp, synthetic construct
  misc_feature 1..23: note = CR-ZF20-R
  source 1..23: mol_type = other DNA
ttaaagcctcagagtgagatcag

SEQ ID NO: 27 - DNA, 25 bp, synthetic construct
  misc_feature 1..25: note = qGmZFP7-F
  source 1..25: mol_type = other DNA
atgatgactccaaacttgaatctgg

SEQ ID NO: 28 - DNA, 25 bp, synthetic construct
  misc_feature 1..25: note = qGmZFP7-R
  source 1..25: mol_type = other DNA
taactttcaactcttcatcaccggg

SEQ ID NO: 29 - DNA, 21 bp, synthetic construct
  misc_feature 1..21: note = qGmActin-F
  source 1..21: mol_type = other DNA
cggtggttctatcttggcatc

SEQ ID NO: 30 - DNA, 22 bp, synthetic construct
  misc_feature 1..22: note = qGmActin-R
  source 1..22: mol_type = other DNA
gtctttcgcttcaataacccta

SEQ ID NO: 31 - DNA, 23 bp, synthetic construct
  misc_feature 1..23: note = sgRNA
  source 1..23: mol_type = other DNA
ggtttgaacttagaccttggtct

SEQ ID NO: 32 - DNA, 151 bp, synthetic construct
  source 1..151: mol_type = other DNA
atgatgactccaaacttgaatctggaaccagaagatgattttaaagccgatcctgacccaggtcctgtttccctggacttgtccctcaacttcaatcccggtgatgaagagttgaaagttacaagtgacactaactgtgaagtaggacctg

SEQ ID NO: 33 - DNA, 23 bp, synthetic construct
  source 1..23: mol_type = other DNA
tctggttccagattcaagtttgg

SEQ ID NO: 34 - DNA, 24 bp, synthetic construct
  source 1..24: mol_type = other DNA
tctggttccagattcaaagtttgg

SEQ ID NO: 35 - DNA, 32 bp, synthetic construct
  source 1..32: mol_type = other DNA
atgactccaaacttgaatctggaaccagaaga

SEQ ID NO: 36 - DNA, 33 bp, synthetic construct
  source 1..33: mol_type = other DNA
atgactccaaactttgaatctggaaccagaaga